SEQUENCE LISTING (4 sequences)

SEQ ID NO: 1 - DNA, 29 bp, Artificial Sequence
  Primer - paired read
aatgatacggcgaccaccgagauctacac

SEQ ID NO: 2 - DNA, 24 bp, Artificial Sequence
  Primer - paired read
  misc_feature (22)..(22): n = 8-oxoguanine
caagcagaagacggcatacganat

SEQ ID NO: 3 - DNA, 20 bp, Artificial Sequence
  Primer - single read
aatgatacggcgaccaccga

SEQ ID NO: 4 - DNA, 21 bp, Artificial Sequence
  Primer - single read
caagcagaagacggcatacga

(19) United States
(12) Patent Application Publication
(10) Pub. No.: US 2022/0145384 A1
(43) Pub. Date: May 12, 2022

(54) DETECTING MATERIALS IN A MIXTURE USING OLIGONUCLEOTIDES

(71) Applicant: ILLUMINA, INC., San Diego, CA (US)

(72) Inventor: Shaun Hunter, San Diego, CA (US)

(73) Assignee: ILLUMINA, INC., San Diego, CA (US)

(21) Appl. No.: 17/511,353

(22) Filed: Oct. 26, 2021

Related U.S. Application Data
(60) Provisional application No. 63/110,655, filed on Nov. 6, 2020.

(51) Int. Cl. C12Q 1/6869; B01L 3/00

(52) U.S. Cl. CPC C12Q 1/6869; B01L 2200/16; B01L 3/527

(57) ABSTRACT

A method of detecting materials in a mixture of materials may include providing materials, and providing oligonucleotides having different sequences than one another. Each of the oligonucleotides may be within, and may correspond to, a respective one of the materials. A mixture of at least two of the materials with one another may be obtained. The mixture may include the oligonucleotides corresponding to those materials. The method may include sequencing the oligonucleotides in the mixture; and detecting the materials corresponding to those oligonucleotides using the sequences of those oligonucleotides.

### CROSS-REFERENCE TO RELATED APPLICATIONS

This application claims the benefit of U.S. Provisional Patent Application No. 63/110,655, filed Nov. 6, 2020 and entitled “Detecting Materials in a Mixture Using Oligonucleotides,” the entire contents of which are incorporated by reference herein.

### SEQUENCE LISTING

The instant application contains a Sequence Listing which has been submitted electronically in ASCII format and is hereby incorporated by reference in its entirety. Said ASCII copy, created on Oct. 15, 2021, is named IP-2035-US-SL.txt and is 1,088 bytes in size.

### BACKGROUND

The detection of specific nucleic acid sequences present in a biological sample has been used, for example, as a method for identifying and classifying microorganisms, diagnosing infectious diseases, detecting and characterizing genetic abnormalities, identifying genetic changes associated with cancer, studying genetic susceptibility to diseases, and measuring response to various types of treatment. A common technique for detecting specific nucleic acid sequences in a biological sample is nucleic acid sequencing.

Nucleic acid sequencing methodology has evolved from the chemical degradation methods used by Maxam and Gilbert and the strand elongation methods used by Sanger. Several sequencing methodologies are now in use which allow for the parallel processing of millions, or even billions, of nucleic acids on a single flow cell. Some platforms include bead-based and microarray formats in which silica beads are functionalized with probes depending on the application of such formats in applications including sequencing, genotyping, or gene expression profiling. Some sequencing systems, whether for “sequencing-by-synthesis” or for genotyping, utilize substrates including a plurality of different reservoirs that carry different reagents for use in sequencing operations.

### SUMMARY

Examples provided herein are related or detecting materials in a mixture using oligonucleotides. Apparatuses using such oligonucleotides also are disclosed.

In some examples, a method of detecting materials in a mixture of materials is provided herein. The method may include providing materials, and providing oligonucleotides having different sequences than one another. Each of the oligonucleotides may be within, and may correspond to, a respective one of the materials. The method may include obtaining a mixture of at least two of the materials with one another. The mixture may include the oligonucleotides corresponding to those materials. The method may include sequencing the oligonucleotides in the mixture; and detecting the materials corresponding to those oligonucleotides using the sequences of those oligonucleotides.

In some examples, the materials include reagents for use in sequencing. In some examples, the materials respectively are provided in reservoirs of a substrate.

In some examples, the sequence of each of the oligonucleotides includes: (i) an index corresponding to the mixture and (ii) a barcode corresponding to the respective material.

In some examples, the sequence of each of the oligonucleotides includes a barcode corresponding to the respective material, and the method further including adding, to each of the oligonucleotides, an index corresponding to the mixture. In some examples, the index is added after obtaining the mixture.

In some examples, the mixture is obtained using a first sequencing system. For example, the mixture may include waste from the first sequencing system. In some examples, the oligonucleotides in the mixture are sequenced using a second sequencing system that is different from the first sequencing system. In some examples, the sequences of the oligonucleotides include adapters that are compatible with the second sequencing system and are incompatible with the first sequencing system.

In some examples, sequencing the oligonucleotides in the mixture includes amplifying the oligonucleotides on a surface to generate respective clusters of amplicons on a surface. In some examples, detecting the materials includes comparing the sequences of the oligonucleotides to stored sequences. In some examples, detecting the materials includes quantifying respective amounts of the oligonucleotides, and correlating the amounts of the oligonucleotides to amounts of the respective materials. In some examples, the oligonucleotides include single-stranded DNA, double-stranded DNA, RNA, LNA, or a sequence of modified nucleotides.

In some examples, wherein each of the materials is independently selected from the group consisting of a liquid, a semi-solid, and a solid. In some examples, the liquid includes a solvent or reagent. In some examples, the solid includes a dry powder or a powder in a liquid. In some examples, the semi-solid includes a gel.

In some examples provided herein is a method of detecting a material in a mixture of materials. The method may include mixing at least two materials with one another to obtain a mixture. Different oligonucleotides may have different sequences than one another and respectively may be disposed within different ones of the materials. The sequences of the oligonucleotides may be used to detect the materials in the mixture.

In some examples, the materials include reagents for use in sequencing. In some examples, the materials respectively are provided in reservoirs of a substrate. In some examples, the sequence of each of the oligonucleotides includes one or both of (i) an index corresponding to the mixture and (ii) a barcode corresponding to the respective material.

In some examples, the mixture is obtained using a first sequencing system. For example, the mixture may include waste from the sequencing system. In some examples, the oligonucleotides in the mixture are sequenced using a second sequencing system that is different from the first sequencing system. In some examples, the sequences of the oligonucleotides include adapters that are compatible with the second sequencing system and are incompatible with the first sequencing system.

In some examples, the materials are detected by comparing the sequences of the oligonucleotides to stored sequences. In some examples, the materials are detected by quantifying respective amounts of the oligonucleotides, and correlating the amounts of the oligonucleotides to amounts of the respective materials. In some examples, the oligonucleotides include single-stranded DNA, double-stranded DNA, RNA, LNA, or a sequence of modified nucleotides.

In some examples, each of the materials is independently selected from the group consisting of a liquid, a semi-solid, and a solid. In some examples, the liquid includes a solvent or reagent. In some examples, the solid includes a dry powder or a powder in a liquid. In some examples, the semi-solid includes a gel.

In some examples herein an apparatus is provided. The apparatus may include a substrate including a plurality of reservoirs. The apparatus may include a plurality of materials, each of the materials being within a respective one of the reservoirs. The apparatus may include a plurality of oligonucleotides having different sequences than one another, each of the oligonucleotides being within a respective one of the materials.

In some examples, the materials include reagents for use in sequencing.

In some examples, the sequence of each of the oligonucleotides includes one or both of (i) an index corresponding to the mixture and (ii) a barcode corresponding to the respective material.

In some examples, the apparatus is for use in a sequencing system to obtain a mixture of two or more of the materials. For example, the mixture may include waste from the sequencing system. In some examples, the sequences of the oligonucleotides include adapters that are incompatible with the sequencing system. In some examples, the oligonucleotides include single-stranded DNA, double-stranded DNA, RNA, LNA, or a sequence of modified nucleotides.

In some examples, each of the materials is independently selected from the group consisting of a liquid, a semi-solid, and a solid. In some examples, the liquid includes a solvent or reagent. In some examples, the solid includes a dry powder or a powder in a liquid. In some examples, the semi-solid includes a gel.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1B schematically illustrate an example apparatus and operations in a process flow for detecting materials in a mixture using oligonucleotides.

FIGS. 2A-2D schematically illustrate example oligonucleotides for use in a process flow such as described with reference to FIGS. 1A-1B.

FIG. 3 schematically illustrates example operations in a process flow for detecting materials in a mixture using oligonucleotides.

FIG. 4 schematically illustrates example operations in another process flow for detecting materials in a mixture using oligonucleotides.

FIG. 5 schematically illustrates another example apparatus and operations in a process flow for detecting materials in a mixture using oligonucleotides.

FIGS. 6A-6C schematically illustrate example measurements in a process flow for detecting liquids in a mixture using oligonucleotides.

### DETAILED DESCRIPTION

Examples provided herein are related or detecting materials in a mixture using oligonucleotides. Apparatuses using such oligonucleotides also are disclosed.

The technology of this application relates to using oligonucleotides that are mixed in small amounts into respective materials for use in qualitatively or quantitatively determine the presence of one or more of such materials in a mixture. The oligonucleotides may be implemented in any suitable environment and any suitable material or materials, e.g., any suitable liquids, semi-solids, or solids. Illustratively, the present oligonucleotides may be used to identify possible sources of apparent contamination, e.g., to detect whether any materials (such as reagents) are intentionally or unintentionally getting mixed into other materials. Additionally, or alternatively, the present oligonucleotides may be used to quantify the effectiveness of a process for mixing two or more materials together, e.g., by detecting the amounts of different materials at different locations in the mixture at one or more times. In various examples, the amounts of the materials may be expressed as absolute amounts in the sample (e.g., as concentrations or volumes), or may be expressed as relative amounts in the sample (e.g., as molar ratios), or may be expressed qualitatively (e.g., as presence or absence). In some examples, the materials may be used in an oligonucleotides sequencing process, in which contamination of one material by another material may detrimentally affect the sequencing process.

In some examples, oligonucleotides having different sequences than each other respectively may be mixed into different materials. Two or more of the materials may be mixed together, and as a result the mixture further may include the oligonucleotides that were mixed into those materials. The oligonucleotides are amplified and then sequenced from a sample of the mixture. Using the sequences of the oligonucleotides that are present, it is determined which materials were mixed together, and the absolute or relative amounts of such materials in the sample. For example, the sequences of the oligonucleotides may include one or more identification sub-sequences which may be arbitrary but unique. For example, a first sub-sequence (which may be referred to herein as a “barcode”) may be unique to the particular material into which the oligonucleotide is mixed, allowing identification of that material when the sequence is detected. A second sub-sequence (which may be referred to herein as an “index”) may be unique to a particular volume of that material, allowing different samples (which may include the same materials as each other, but from different volumes of those materials) to be pooled together for sequencing purposes while allowing the identification of the particular volumes from which the materials in those samples originated. The second sub-sequence may be added after taking the sample of the mixture, and may be omitted, e.g., if samples are not being pooled together. The oligonucleotides also may include adapters for use in amplification. In examples in which the oligonucleotides are being mixed into reagents being used in a first sequencing process, the adapters of those oligonucleotides may be orthogonal to those that may be used in the first sequencing process, such that the oligonucleotides themselves may not be amplified or sequenced in the first sequencing process and instead are amplified and sequenced in a second sequencing process that may be performed separately from the first sequencing process, and may be performed on a different sequencing system from the first sequencing process. However, it will be appreciated that the present technology is not limited to use in sequencing reagents, and may be used to detect any number of materials in any given mixture of materials.

First, some terms used herein will be briefly explained. Then, some example methods for detecting materials in a mixture using oligonucleotides, and associated apparatuses, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Example polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” refers to a polynucleotide to which nucleotides may be added via a free 3′ OH group. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer. A primer may be coupled to a substrate. Primers that are “complementary” to one another may hybridize to one another along substantially their entire lengths, whereas primers that are “orthogonal” with one another substantially do not hybridize with one another, nor do their amplicons.

In some examples, primers are P5 or P7 primers that are commercially available from Illumina, Inc. P5 and P7 primers are nonlimiting examples of primers that are orthogonal to one another. The P5 and P7 primer sequences may have the following sequences, in some examples:

### Paired Read Set:

P5:
(SEQ ID NO: 1)
5′-AATGATACGGCGACCACCGAGAUCTACAC-3′
P7:
(SEQ ID NO: 2)
5′-CAAGCAGAAGACGGCATACGAG*AT-3′

### Single Read Set:

P5:
(SEQ ID NO: 3)
5′-AATGATACGGCGACCACCGA-3′
P7:
(SEQ ID NO: 4)
5′-CAAGCAGAAGACGGCATACGA3′

where G* is G or 8-oxoguanine.

In some examples, the attached oligonucleotides (such as primers or P5 or P7 primers) include a linker or spacer at the 5′ end. Such linker or spacer may be included in order to permit chemical or enzymatic cleavage, or to confer some other desirable property, for example to enable covalent attachment to a polymer or a solid support, or to act as spacers to position the site of cleavage an optimal distance from the solid support. In certain cases, 10 spacer nucleotides may be positioned between the point of attachment of the P5 or P7 primers to a polymer or a solid support. In some examples, polyT spacers are used, although other nucleotides and combinations thereof can also be used. In one example, the spacer is a 6T to 10T spacer. In some examples, the linkers include cleavable nucleotides including a chemically cleavable functional group such as a vicinal diol or allyl T.

As used herein, the term “amplicon,” when used in reference to a polynucleotide, is intended to means a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface comprises a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may comprise a single material or a plurality of different materials. Substrates may be composites or laminates. In some examples, the substrate comprises an organo-silicate material. Substrates may be flat, round, spherical, rod-shaped, or any other suitable shape. Substrates may be rigid or flexible. In some examples, a substrate is a bead or a flow cell.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of wells (e.g., microwells or nanowells) on glass, silicon, plastic or other suitable material(s) with patterned, covalently-linked gel such as poly(N-(5-azidoacetamidylpentyl) acrylamide-co-acrylamide) (PAZAM). The process creates gel pads used for sequencing that may be stable over sequencing runs with a large number of cycles. The covalent linking of the polymer to the wells may be helpful for maintaining the gel in the structured features throughout the lifetime of the structured substrate during a variety of uses. However in many examples, the gel need not be covalently linked to the wells. For example, in some conditions silane free acrylamide (SFA) which is not covalently attached to any part of the structured substrate, may be used as the gel material.

In particular examples, a structured substrate may be made by patterning a suitable material with wells (e.g. microwells or nanowells), coating the patterned material with a gel material (e.g., PAZAM, SFA or chemically modified variants thereof, such as the azidolyzed version of SFA (azido-SFA)) and polishing the surface of the gel coated material, for example via chemical or mechanical polishing, thereby retaining gel in the wells but removing or inactivating substantially all of the gel from the interstitial regions on the surface of the structured substrate between the wells. Primers may be attached to gel material. A solution including a plurality of target polynucleotides (e.g., a fragmented human genome or portion thereof) may then be contacted with the polished substrate such that individual target polynucleotides will seed individual wells via interactions with primers attached to the gel material; however, the target polynucleotides will not occupy the interstitial regions due to absence or inactivity of the gel material. Amplification of the target polynucleotides may be confined to the wells because absence or inactivity of gel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An example patterned substrate having these characteristics is the etched substrate used in connection with BEAD ARRAY technology (Illumina, Inc., San Diego, Calif.).

In some examples, a substrate forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, Calif.).

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “sequencing system” refers to a system that is configured to determine the sequence of polynucleotides. A variety of sequencing systems are commercially available. Illustratively, a sequencing system may be or include the iSEQ™ 100 Sequencing System, commercially available from Illumina, Inc. (San Diego, Calif.). The iSEQ™ 100 Sequencing System is a benchtop system that performs sequencing-by-synthesis using a prefilled cartridge that includes reservoirs storing different sequencing reagents. Other nonlimiting examples of sequencing systems include the cBot 2, NovaSeq 6000, and MiniSeq systems commercially available from Illumina, Inc., as well as sequencing systems from other sources.

As used herein, terms such as “mixing” and “mixed” are intended to mean that materials are combined with one another in such a manner that each of the materials becomes distributed throughout the “mixture” resulting from such combination. The distribution of any suitable number of such materials may be homogeneous (that is, evenly distributed) throughout the mixture, such that any given portion of that mixture may include substantially the same concentration of such material(s) as any other given portion of that mixture. Additionally, or alternatively, the distribution of any suitable number of such materials may be heterogeneous (that is, unevenly distributed) throughout the mixture, such that any given portion of that mixture may include a different concentration of such material(s) as one or more other given portions of that mixture. Within a given mixture, one or more materials may be homogeneous and one or more materials may be heterogeneous. Each material in a given mixture may retain its own chemical identity (that is, does not chemically react with any other materials in the mixture). Alternatively, two or more of the materials in a given mixture may chemically react with one another to form one or more new materials.

Any suitable type(s) of materials may be mixed together to form a mixture, such as any suitable combination of liquids, solids, and/or semi-solids. The extent to which the mixture is homogeneous or heterogeneous may depend on the particular materials being mixed, e.g., based on the extent (if any) to which each material is soluble in any of the other materials in the mixture. For example, miscible liquids may be mixed to form a homogeneous mixture, whereas immiscible liquids may be mixed to form a heterogeneous mixture, such as an emulsion including droplets of one liquid dispersed in another liquid. Additionally, or alternatively, a liquid may be mixed with a solid, such as a particulate solid (a solid having particles with dimensions of, for example, about 1 nm to about 1 mm, or about 10 nm to about 100 m, or about 100 nm to about 10 m). When a solid dissolves in the liquid, the resulting mixture may be homogeneous, whereas when a solid becomes suspended in the liquid and does not dissolve in the liquid (e.g., forms a colloidal suspension of solid particles within the liquid), the resulting mixture may be heterogeneous. Nonlimiting examples of liquids include solvents (such as water or organic solvents) and reagents (such as chemical or biological species that may react with one or more other chemical or biological species).

Additionally, or alternatively, a liquid may be mixed with a semi-solid such as a “gel”, e.g., a material that includes a three-dimensionally cross-linked structure (e.g., a cross-linked polymer, cross-linked network of colloidal particles, or cross-linked network of nanoparticles or nanostructures) with liquid disposed within the structure. When a gel dissolves in the liquid, the resulting mixture may be homogeneous, whereas when a gel becomes suspended in the liquid and does not dissolve in the liquid (e.g., forms a colloidal suspension of gel particles within the liquid), the resulting mixture may be heterogeneous. Additionally, or alternatively, a solid may be mixed with a semi-solid such as a gel. When a solid dissolves in the gel, then the resulting mixture may be homogeneous, whereas when a solid becomes suspended in the gel and does not dissolve in the gel (e.g., forms a suspension of solid particles within the gel), then the resulting mixture may be heterogeneous. Additionally, or alternatively, a semi-solid such as a gel may be mixed with another semi-solid such as a gel. When gels are miscible, e.g., one gel dissolves in the other the gel, the resulting mixture may be homogeneous, whereas when one gel becomes suspended in the other gel and does not dissolve in the gel (e.g., forms a suspension of gel particles within the other gel), the resulting mixture may be heterogeneous. Nonlimiting examples of gels include hydrogels, organogels, and nanocomposite hydrogels.

Additionally, or alternatively, a solid may be mixed together with one or more other solids. Non limiting examples of solids that may be mixed together are dry powders (e.g., agglomerations or lyophilized media such as microspheres). Each of the solids may be flowable, that is, able to flow in such a manner as to be mixable with one another. Depending on the manner and extent to which solids are mixed with one another, a mixture of solids may be either homogeneous or heterogeneous. Nonlimiting examples of solids include polymers, glasses, semiconductors, metals, salts, and ceramics.

Additionally, oligonucleotides may be mixed into any suitable material(s), e.g., any suitable liquid, solid, and/or semi-solid. For example, an oligonucleotide may be dissolved into a liquid, may be dissolved into a gel, may be incorporated into a solid, or may coat the outer surface of a particulate solid. As such, mixtures of any combination of such materials also will include the oligonucleotides that were mixed into such materials. Accordingly, in a manner such as described elsewhere herein, any such oligonucleotide may be amplified, sequenced, and used to detect the material into which that oligonucleotide was mixed.

Mixtures of different materials with one another may be obtained using any suitable using any suitable process or mixer, e.g., using convection, stirring, agitation, turbulence, diffusion, or laminar mixing. In one nonlimiting example, materials may become mixed together in a waste receptacle of a system (such as a sequencing system) as that system uses and then disposes of those materials after such use.

Detecting Materials in a Mixture Using Oligonucleotides, and Associated Apparatuses

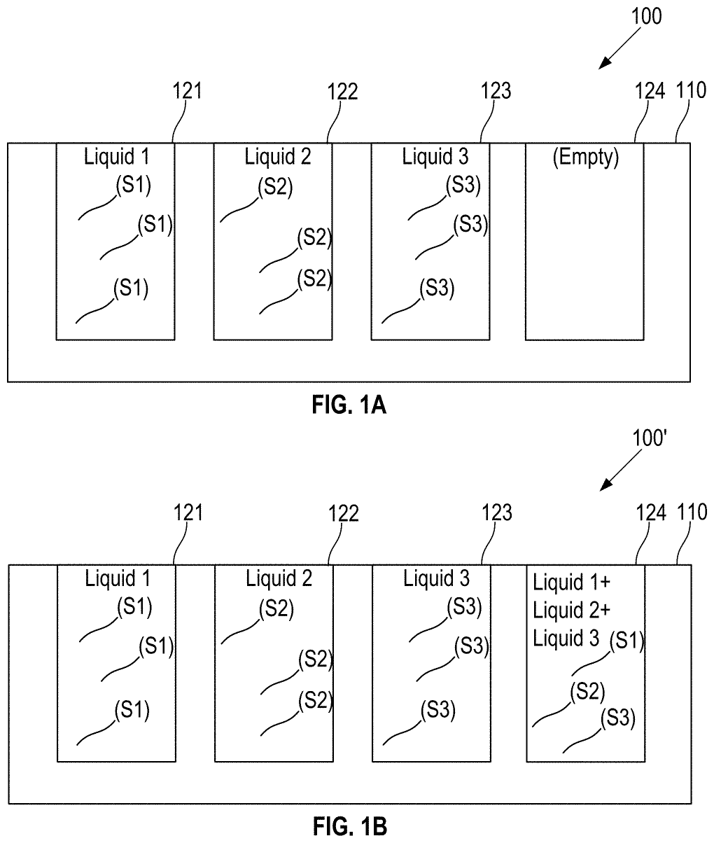

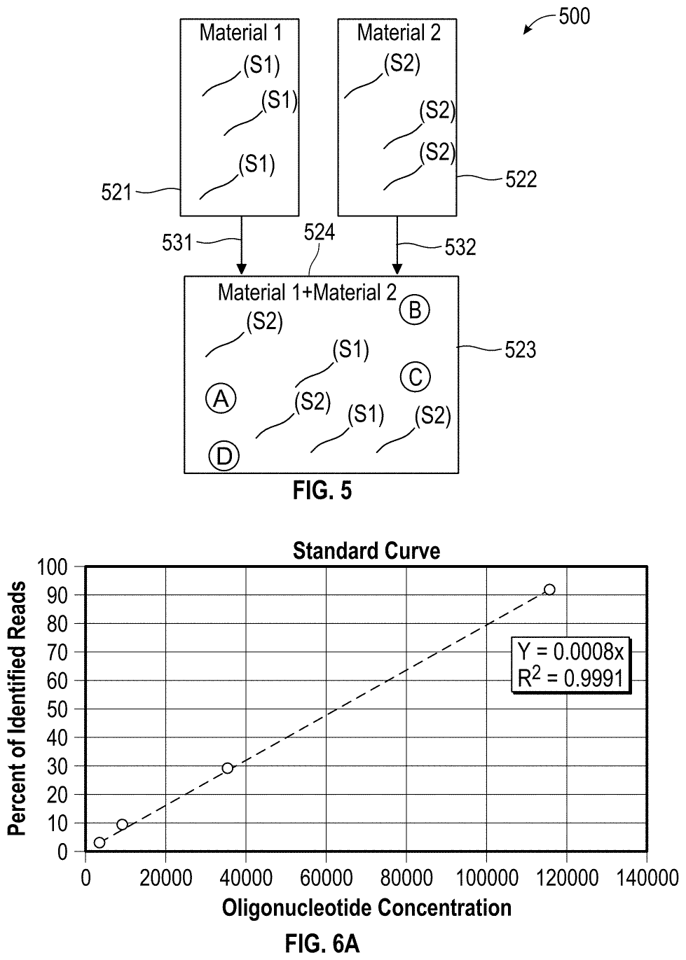

As noted above and as described in greater detail below, the present apparatuses and methods may be used to detect one or more materials in a mixture of materials. The materials that may be detected using the present subject matter include any suitable combination of liquids, semi-solids, and solids. For example, FIGS. 1A-1B schematically illustrate an example apparatus and operations in a process flow for detecting materials in a mixture using oligonucleotides. Apparatus 100 illustrated in FIG. 1A may include a substrate 110 including comprising a plurality of reservoirs, e.g., reservoirs 121, 122, 123, 124. In the nonlimiting example illustrated in FIG. 1A, reservoirs 121, 122, 123, 124 may include wells that are provided within a common, integrally formed substrate 110 as one another. However, it will be appreciated that one or more of reservoirs 121, 122, 123, 124, and indeed all of reservoirs 121, 122, 123, 124, may be physically separated from one another and need not be formed in a common substrate as one another. For example, in a manner such as described with reference to FIG. 5, the reservoirs may be physically separated from one another.

In the nonlimiting example illustrated in FIG. 1A, apparatus 100 may include plurality of materials, e.g., Liquid 1, Liquid 2, and Liquid 3, although it will be understood that any other suitable type(s) of material(s) may be used. Each of the materials, e.g., liquids, may be within a respective one of the reservoirs. For example, Liquid 1 is stored within reservoir 121, Liquid 2 is stored within reservoir 122, and Liquid 3 is stored within reservoir 123. In some examples, one or more of Liquid 1, Liquid 2, and Liquid 3 include reagents for use in sequencing. Illustratively, Liquid 1, Liquid 2, and Liquid 3 may include sequencing reagents selected from the group consisting of: polymerase, labeled nucleotides, wash buffers, cleavage reagent, and other enzymatic reagents. Illustratively, apparatus 100 may be for use in a sequencing system to obtain the mixture of two or more of the materials, e.g., liquids. Illustratively, the sequencing system may be or include the iSEQ™ 100 Sequencing System, commercially available from Illumina, Inc. (San Diego, Calif.). The iSEQ™ 100 Sequencing System is a benchtop system that performs sequencing-by-synthesis using a prefilled cartridge that includes reservoirs storing different sequencing reagents in a manner similar to that of apparatus 100 illustrated in FIGS. 1A-1B. However, it will be appreciated that apparatus 100 suitably may be adapted for use with any other sequencing system, such as the cBot 2, NovaSeq 6000, or MiniSeq systems commercially available from Illumina, Inc., sequencing systems from other sources, and indeed any other system in the biological, pharmaceutical, chemical, engineering, or other technology area in which one or more materials is used.

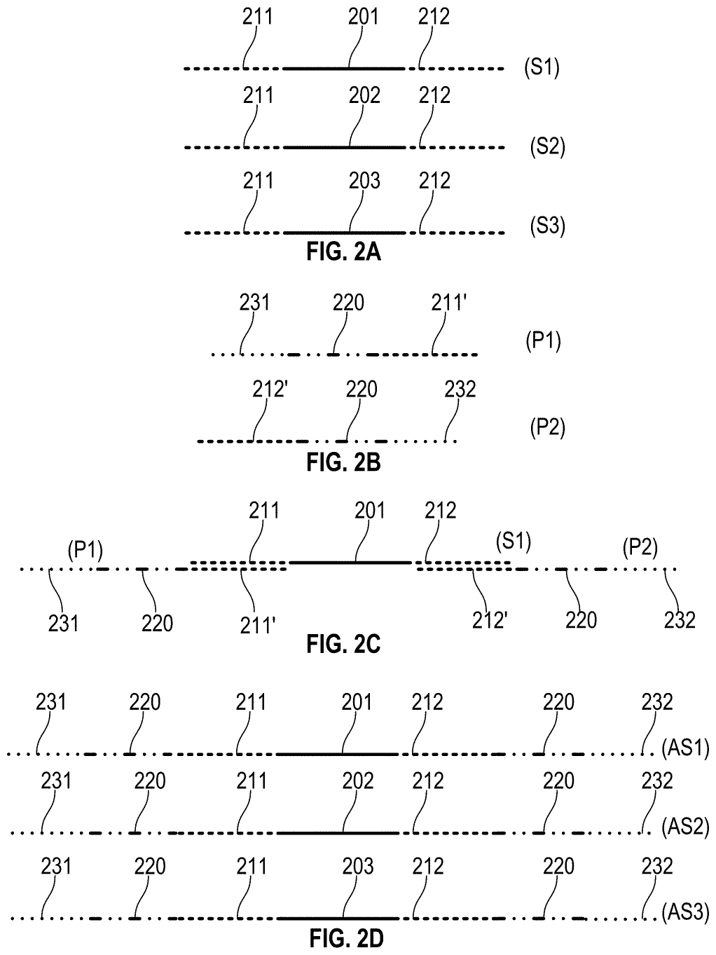

As illustrated in FIG. 1A, apparatus 100 may include a plurality of oligonucleotides having different sequences than one another, each of the oligonucleotides being within a respective one of the materials. For example, oligonucleotide S1 having a first sequence may be within Liquid 1 in reservoir 121, oligonucleotide S2 having a second, different sequence may be within Liquid 2 in reservoir 122, and oligonucleotide S3 having a third, still different sequence may be within Liquid 3 in reservoir 123. In a manner such as described in greater detail below with reference to FIGS. 2A-2D, oligonucleotides S1, S2, and S3 may include adapters that can be used to amplify the oligonucleotides, and also may include unique sequences that can be used to respectively detect Liquid 1, Liquid 2, and Liquid 3 using the amplified sequences of such oligonucleotides. In some examples, one or more of the oligonucleotides may be modified for compatibility with the material in which it is provided. Illustratively, an oligonucleotide for use in a liquid including an exonuclease may be modified to include a phosphorothioate bond at the 3′ end to inhibit degradation by the exonuclease.

At the particular time illustrated in FIG. 1A, reservoir 124 may be empty. In comparison, at the particular time illustrated in FIG. 1B, reservoir 124 may store a mixture of two or more of Liquid 1, Liquid 2, and Liquid 3 in a manner such as illustrated in FIG. 1B. In some examples, reservoir 124 may be used to collect waste from processes (e.g., sequencing processes) using Liquid 1, Liquid 2, and/or Liquid 3, and a result of such collection may include some of each of the materials. In other examples, Liquid 1, Liquid 2, and/or Liquid 3 may be mixed together within reservoir 124, and the mixture used in a process (e.g., a sequencing process). The mixture within reservoir 124 may include the oligonucleotides corresponding to those materials, e.g., may include S1, S2, and/or S3.

A sample may be obtained of the mixture of at least two of the materials with one another, e.g., a sample of the mixture of Liquid 1, Liquid 2, Liquid 3 illustrated in FIG. 1B, and such sample similarly may include oligonucleotides S1, S2, and S3 as in the nonlimiting, illustrated example. The sample may be of any appropriate size, e.g., may have a volume of about 1 nL to about 1 mL, or a volume of about 10 nL to about 100 μL, or a volume of about 100 nL to about 10 μL, or a volume of about 100 nL to about 1 μL, or a volume of about 10 nL to about 100 nL, or a volume of about 1 nL to about 10 nL. The oligonucleotides in the sample of the mixture may be sequenced, and the materials corresponding to those oligonucleotides may be detected using the sequences of those oligonucleotides. For example, the oligonucleotides within the sample of the mixture may be amplified in a manner such as described with reference to FIGS. 2A-2D, and then may be sequenced. The resulting detected sequences of oligonucleotides S1, S2, S3 respectively may be correlated with the materials 121, 122, 123 which are known to include those oligonucleotides in a known concentration. For example, detection circuitry may include a computer-readable medium and a processor configured to use information and instructions stored in the computer-readable medium. The information stored may include (i) oligonucleotide sequences, (ii) identification of the materials into which the oligonucleotides having those sequences respectively are mixed, and (iii) the concentration at which the oligonucleotides are mixed into those materials. The instructions may cause the processor to (i) compare the detected sequences of S1, S2, and S3 to the stored oligonucleotide sequences, (ii) using such comparison, determine which of the stored oligonucleotide sequences correspond to the detected oligonucleotide sequences, and (iii) for each of the stored oligonucleotide sequences determined to correspond to a detected oligonucleotide sequence, use the stored information to determine which material(s) are present in the sample. Accordingly, from the presence of the sequence of a given oligonucleotide, the presence of the material may be detected. In this, oligonucleotide S1, S2, and S3 may be used to detect presence of respective materials Liquid 1, Liquid 2, and Liquid 3 within the mixture.

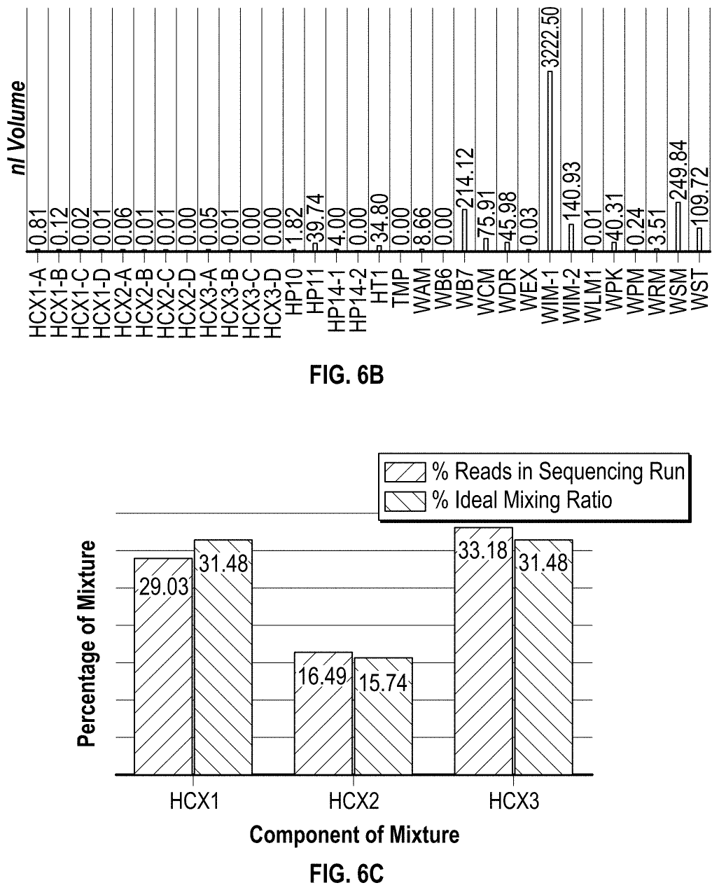

Additionally, from the amount of the sequence of a given oligonucleotide, the absolute or relative amount of the material may be determined. For example, a standard curve such as described below with reference to FIG. 6A may be prepared ahead of time that correlates the amount of identified reads of a particular sequence in a material to the concentration of the oligonucleotide in that material, and the slope and offset of that curve may be stored in the detection circuitry. For example, a standard curve may be generated and stored for each of the oligonucleotides using an “internal ladder” which also may be referred to as a concentration ladder. More specifically, different materials may be prepared, each of which includes multiple of the oligonucleotides, each of which oligonucleotides is at different known concentration in each of the materials. For example, the different materials may be prepared with a specified concentration of each of the oligonucleotides, or the concentrations of each of the oligonucleotides may be determined and then stored prior to exposure for contamination or mixing. The concentrations used to generate the standard curve may range, for example, from about 1 pM to about 1000 pM, or about 10 pM to about 1000 pM, or about 10 pM to about 100 pM.

The detection circuitry (e.g., processor on board the sequencing system or other type of system) may use such standard curve (stored within a computer-readable medium) to calculate the concentration of an oligonucleotide in a sample of a material from the amount of identified reads of that oligonucleotide's sequence from that sample, for example by comparing the amount of identified reads of that oligonucleotide's sequence from the sample to the amounts of identified reads in the standard curve for that oligonucleotide, and identifying a concentration of oligonucleotide in the standard curve that corresponds to that amount of identified reads in the standard curve, which concentration corresponds to the concentration of the oligonucleotide in the sample. The concentration of the material in the sample then may be calculated using the determined concentration of the oligonucleotide in the sample and the known concentration of the oligonucleotide in the material. The detection circuitry may express the amount of the material in the sample as an absolute amount (e.g., as concentration, or as a volume by multiplying the concentration by the amount of sample that was analyzed), or may express the amount of the material in the sample as a relative amount (e.g., as molar ratios, which may be calculated by dividing the concentration of one material in the sample by the concentration of one or more other materials in the sample). The materials may be detected at relatively low volumes and amounts in the sample, e.g., in volumes of about 0.1-500 nL, of about 0.1-100 nL of about 0.1-10 nL, of about 0.1-1 nL.

FIGS. 2A-2D schematically illustrate example oligonucleotides for use in a process flow such as described with reference to FIGS. 1A-1B. Each oligonucleotide may include a sub-sequence (which may be referred to herein as a “barcode”) that is unique to the particular material into which the oligonucleotide is mixed, allowing identification of that material when the sequence is detected. Each oligonucleotide also may include a sub-sequence (which may be referred to herein as an “index”) that is unique to a particular volume of that material, allowing different samples (which may include the same materials as each other, but from different volumes of those materials) to be pooled together for sequencing purposes while allowing the identification of the particular volumes from which the materials in those samples originated. In the illustrated example, oligonucleotide S1 includes barcode 201 corresponding to respective Liquid 1, first adapter 211, and second adapter 212. Similarly, oligonucleotide S2 includes barcode 202 corresponding to respective Liquid 2, first adapter 211, and second adapter 212. Similarly, oligonucleotide S3 includes barcode 203 corresponding to respective Liquid 3, first adapter 211, and second adapter 212. In some examples, oligonucleotides S1, S2, and S3 may include single-stranded DNA, double-stranded DNA, RNA, LNA, or a sequence of modified nucleotides. Oligonucleotides S1, S2, and S3 may include the same type of nucleotides as one another (e.g., all may include DNA, RNA, LNA, or the same type of modified nucleotides), or may include different types of nucleotides as one another.

First adapters 211 and second adapters 212 may have the same sequences as one another, and may be used in a process to amplify oligonucleotides S1, S2, and S3 in a sample of a mixture. However, first and second adapters 211, 212 may be incompatible with the sequencing system in which the materials are used and thus may inhibit or prevent amplification of oligonucleotides S1, S2, and S3, but are compatible with the sequencing system in which the oligonucleotides S1, S2, S3 are amplified and detected. That is, first and second adapters 211, 212 may be orthogonal to primers that are used in the sequencing system in which the materials are used, and may be complementary to primers that are used in the sequencing system in which the oligonucleotides S1, S2, S3 are amplified and detected. In one nonlimiting example, first and second adapters 211, 212 are NEXTERA® adapters commercially available from Illumina, Inc., and may be added to barcodes 201, 202, 203 using a tagmentation process in which transposomes are used to cleave DNA into fragments and add adapters to both ends of the fragmented DNA. Alternatively, oligonucleotides may be prepared or commercially ordered having the desired sequence, e.g., including the barcode and adapters.

Barcodes 201, 202, and 203 may have different sequences than one another, and as such may be used to respectively detect and distinguish the materials, e.g., Liquid 1, Liquid 2, and Liquid 3, from one another. As such, although some portions of the sequences of oligonucleotides S1, S2, and S3 may be the same as one another (e.g., first adapters 211 and second adapters 212), the sequences of the oligonucleotides may be considered to be different than one another because barcodes 201, 202, 203 are different than one another. The particular sequences used in barcodes 201, 202, 203 need not have any particular relationship to one another other than being different than one another, and indeed may be arbitrary. Additionally, the lengths of barcodes 201, 202, 203 may include any suitable number of nucleotides and need not necessarily have the same number of nucleotides as each other, e.g., may include from about 1 to about 200 nucleotides, or about 2 to about 150 nucleotides, or about 5 to about 100 nucleotides, or about 5 to about 50 nucleotides, or about 10 to about 40 nucleotides, or about 20 to about 30 nucleotides. It should be noted that the greater the number of nucleotides in barcodes 201, 202, 203, the greater the different number of different sequences may be prepared and thus the greater the different number of materials may be detected using such sequences. In one nonlimiting, purely illustrative example, barcodes 201, 202, 203 are or include different fragments of the single-stranded DNA genome of PhiX (an icosahedral, nontailed bacteriophage). In examples using NEXTERA® adapters, the PhiX fragments may be generated, and have adapters 211, 212 added thereto, during the tagmentation process.

Additionally, or alternatively, in some examples, the sequence of each of the oligonucleotides S1, S2, S3 includes an index corresponding to the mixture. Such an index may be useful, for example, for pooling together mixtures that are obtained from different samples, where the index corresponds to each such sample. In some examples, the index may be included within oligonucleotides S1, S2, and S3 prior to adding the oligonucleotides to their respective materials. Alternatively, the index may be added to oligonucleotides S1, S2, and S3 after obtaining the sample of the mixture. For example, FIG. 2B illustrates amplification primers P1 and P2 that may be used to add index 220 to oligonucleotides S1, S2, and S3. Amplification primer P1 includes primer 231 that is complementary to a first surface primer in the sequencing system on which oligonucleotides S1, S2, and S3 are to be sequenced, index sequence 220 corresponding to the mixture, and primer 211′ that is complementary to adapter 211 of oligonucleotides S1, S2, and S3. Amplification primer P2 includes primer 232 that is complementary to a second surface primer in the sequencing system on which oligonucleotides S1, S2, and S3 are to be sequenced, index sequence 220 corresponding to the mixture, and primer 212′ that is complementary to adapter 212 of oligonucleotides S1, S2, and S3. Amplification primers P1 and P2 may be mixed with the sample of the mixture including oligonucleotides S1, S2, and S3 and used both to amplify each of those oligonucleotides to a concentration that readily may be sequenced (e.g., using polymerase chain reaction), and to provide index sequence 220 within each of the resulting amplicons.

For example, in a manner such as illustrated in FIG. 2C, primer 211′ of amplification primer P1 may hybridize to adapter 211 of an oligonucleotide S1, S2, S3, primer 212′ of amplification primer P2 my hybridize to adapter 212 of that oligonucleotide. An amplification process then may be performed which extends oligonucleotide S1 to include two instances of a complement of index 220 as well as complements of primer 231 and primer 232; and extends amplification primers P1 and P2 so as to meet one another and form a contiguous oligonucleotide including a complement of barcode 201. Each of the resulting oligonucleotides then may be further amplified (e.g., using polymerase chain reaction). Such a process may occur concurrently for oligonucleotides S2 and S3, thus generating amplicons of each of the oligonucleotides in the mixture. For example, FIG. 2D illustrates amplicon AS1 of oligonucleotide S1, amplicon AS2 of oligonucleotide S2, and amplicon AS3 of oligonucleotide S3. Oligonucleotides S1, S2, and S3 may be selected so as to amplify at approximately the same rate as one another, and as such the concentrations of the amplicons may be expected to be proportional to the concentrations of the oligonucleotides S1, S2, and S3. In some examples, primers 231 and 232 may be hybridized to corresponding surface primers in a sequencing system, and used to further amplify the amplicons at the surface to form clusters that may be suitably sequenced, e.g., using sequencing-by-synthesis.

When amplicons AS1, AS2, and AS3 are sequenced, the sequences may include both the respective barcodes 201, 202, and 203 of oligonucleotides S1, S2, and S3 respectively corresponding to the materials, e.g., Liquid 1, Liquid 2, and Liquid 3, in which those oligonucleotides had been provided, and index 220 corresponding to the mixture that included those oligonucleotides. A different index may be included in oligonucleotides that are obtained from other mixtures, independently of whether any of the oligonucleotides in those mixtures are from the same materials as one another. As such, when the resulting amplicons of those oligonucleotides are sequenced, the sequence may include respective barcodes respectively corresponding to the materials in which those oligonucleotides had been provided, and an index corresponding to the mixture that included those oligonucleotides. If desired, the amplicons from different mixtures may be pooled together and sequenced together in a common process, and the sequence of each such amplicon may be used to identify both the material to which the corresponding oligonucleotide had been added (using the barcode), and the mixture of materials that included those oligonucleotides (using the index). Such pooling may obviate the need to run separate (e.g., sequential) sequencing processes, and allow the detection of materials in a plurality of mixtures all at the same time as one another.

Note that an index need not necessarily be added to the oligonucleotides from the mixture, e.g., in a manner such as described with reference to FIGS. 2B and 2C. Instead, the oligonucleotides may be amplified using amplification primers P1 and P2 that omit index 220 and attach primers 231 and 232 that may be hybridized to surface primers in the sequencing system, or primers 211, 212 of oligonucleotides S1, S2, and S3 may be directly hybridized to surface primers in the sequencing system such that the oligonucleotides are amplified at that surface.

It will be appreciated that although FIGS. 1A-1B and 2A-2C may describe the use of a mixture of three materials, a mixture of any suitable number of materials similarly may be processed so as to detect the materials in such mixture using the sequences of oligonucleotides provided within those materials. It also will be appreciated that not every material need necessarily include the present oligonucleotide, and not every mixture need include multiple or (even one) of the materials that include the present oligonucleotide. For example, one or more of the materials may include the present oligonucleotide, and a given mixture may or may not include that material and thus may or may not include the corresponding oligonucleotide. From the absence of the oligonucleotide in the mixture, it may be determined that the corresponding material substantially was not present in the mixture.

It will also be appreciated that the concentration of a given material may not necessarily be uniform throughout an entire volume of a mixture. As provided herein, sequences of oligonucleotides may be used to determine the amount or concentration of a given material at any given sub-volume within a volume of a mixture. For example, FIG. 5 schematically illustrates another example apparatus and operations in a process flow for detecting materials in a mixture using oligonucleotides. Apparatus 500 illustrated in FIG. 5 includes first material reservoir 521 storing Material 1 having first oligonucleotide S1 therein, second material reservoir 522 storing Material 2 having second oligonucleotide S2 therein, and third material reservoir 533 receiving Material 1 from first reservoir 521 via conduit 531 and receiving Material 2 from second reservoir 522 via conduit 532. Third reservoir 523 may receive any suitable number of materials from any suitable number of reservoirs via any suitable structures, and any suitable number of such materials may include respective oligonucleotides. Material 1 and Material 2 may be mixed with one another within third reservoir 523 using any suitable process or mixer, e.g., using convection, stirring, agitation, turbulence, diffusion, or laminar mixing. Through such mixing, Material 1 and Material 2 may be distributed throughout third reservoir 523, and as such oligonucleotides S1 and S2 may be distributed throughout third reservoir 523.

However, the mixture of Material 1 and Material 2 within third reservoir 523 may not necessarily be homogeneous at all times (or indeed, at any time), and the amounts of S1 and S2 at different volumes within third reservoir 523 may vary accordingly. Indeed, at any given time, the relative proportion (e.g., molar ratio) of Material 1 to Material 2 in two given volumes within the mixture may be different than at another given volume in the mixture. Similarly, at two different given times, the relative proportion (e.g., molar ratio) of Material 1 to Material 2 even at the same volume within the mixture may be different at different times. So as to assess the extent to which Material 1 and Material 2 are mixed together at a particular sub-volume of the mixture at a particular time, a sample may be obtained from that sub-volume at that time. For example, samples may be obtained corresponding to the sub-volumes denoted A, B, C, and D within FIG. 5. The samples may be obtained at any desired time(s), e.g., at different times than one another, or at the same time as one another. Illustratively, the samples may include predefined sub-volumes that are respectively disposed at locations at which a suitable sampling device, such as a pipette (not specifically illustrated), is actuated. The oligonucleotides within each of the samples may be sequenced in a manner such as described with reference to FIGS. 1A-1, and the amounts of those oligonucleotides used to determine the amounts of Material 1 and Material 2 within each of the samples.

For example, batches of materials with materials with known or determined amounts of oligonucleotides may be mixed using the design or process of interest. The ratio of the oligonucleotides detected in a sample of the mixture is expected to be proportional to the ratio of the materials within that sample, thus providing a quantitative measurement of a dispense or mixing event. Repeating such a measurement multiple times in different samples of the same run and/or in different runs may provide a measure of the variance within a given run, or from run to run. As such, in the development of the design or process, the present subject matter provides a sophisticated measurement tool to assess the mixing results. For example, it may be used to show which components are not present in the expected amount at a particular region of a mixing vessel or reaction vessel, and thus identify design or process features that may be improved. Following changes in the design or process, the measurements may be repeated so as to evaluate the effect of those changes relative to the prior iteration. In other examples, the oligonucleotides may be used to confirm wash protocols or carry-over contamination from batch to batch or if the equipment is used for different reagents or products.

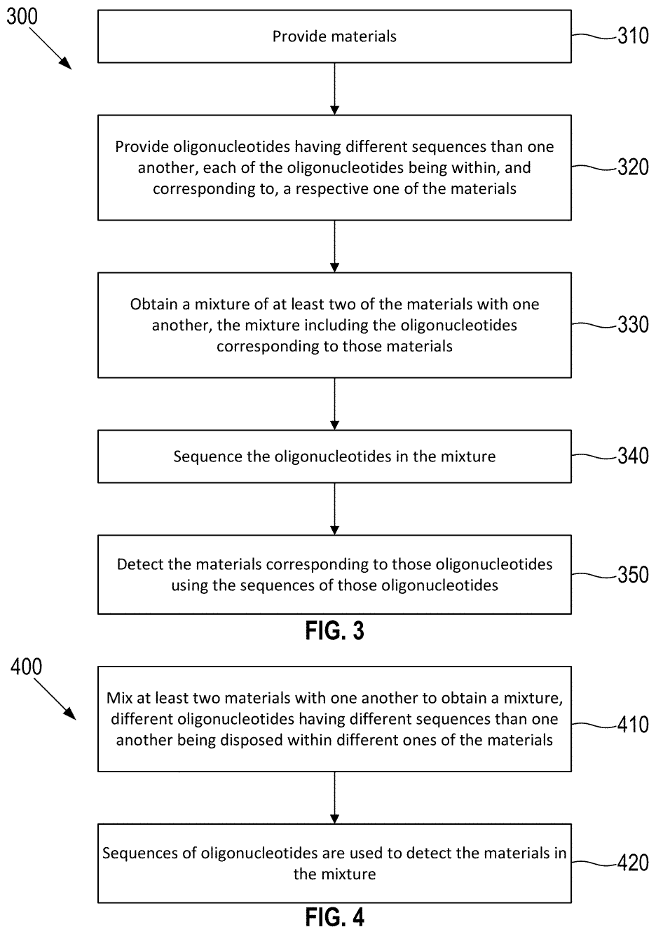

It further will be appreciated that any suitable order and sequence of operations may be used to detect materials in a mixture using oligonucleotides. For example, FIG. 3 schematically illustrates example operations in a process flow for detecting materials in a mixture using oligonucleotides. Process flow 300 illustrated in FIG. 3 may include providing materials (operation 310). For example, any suitable number of materials may be provided in separate reservoirs in a manner such as described with reference to FIG. 5, or may be provided in reservoirs that are provided in a common substrate in a manner such as described with reference to FIGS. 1A-1B. The materials independently may be selected from the group consisting of a liquid, a semi-solid, and a solid. In one nonlimiting example, at least one of the materials includes a reagent for use in a sequencing process.

Process flow 300 illustrated in FIG. 3 also may include providing oligonucleotides having different sequences than one another, each of the oligonucleotides being within, and corresponding to, a respective one of the materials (operation 320). For example, each of the materials may include a known concentration of an oligonucleotide having a sequence that is different from the sequences of oligonucleotides included within any other of the materials. Note that not every material necessarily needs to include such an oligonucleotide. In a manner such as described with reference to FIGS. 2A-2D, the sequence of each of the oligonucleotides may include a barcode corresponding to the respective material. In some examples, an index corresponding to the mixture may be included within, or added to, each of the oligonucleotides. In some examples, the oligonucleotides include single-stranded DNA, although it will be appreciated that other types of oligonucleotides suitably may be used.

Process flow 300 illustrated in FIG. 3 also may include obtaining a mixture of at least two of the materials with one another, the mixture comprising the oligonucleotides corresponding to those materials (operation 330). In some examples, such as described with reference to FIGS. 1A-1B, the mixture may be obtained using a first sequencing system. The mixture may, but need not necessarily, include waste from the first sequencing system. In other examples, such as described with reference to FIG. 5, the mixture may be obtained using mixers provided within a reservoir receiving the materials from other reservoirs.

Process flow 300 illustrated in FIG. 3 also may include sequencing the oligonucleotides in the mixture (operation 340). Sequencing the oligonucleotides in the mixture may include amplifying the oligonucleotides on a surface to generate respective clusters of amplicons on a surface. For example, the oligonucleotides may be amplified in a manner such as described with reference to FIGS. 2A-2D, e.g., so as to add an index and/or to add primers that are complementary to surface primers in a sequencing system used to sequence the oligonucleotides. Such sequencing system may be different from a sequencing system in which the fluids themselves are used. The sequences of the oligonucleotides may, in some examples, adapters that are compatible with the sequencing system being used to sequence the oligonucleotides, and are incompatible with the sequencing system in which the fluids themselves are used.

Process flow 300 illustrated in FIG. 3 also may include detecting the materials corresponding to those oligonucleotides using the sequences of those oligonucleotides (operation 350). For example, the sequences of the oligonucleotides may be compared to stored sequences corresponding to the oligonucleotides provided within the respective materials. In some examples, respective amounts of the oligonucleotides may be quantified, and such amounts of the oligonucleotides correlated to amounts of the respective materials, e.g., using a standard curve such as described elsewhere herein.

FIG. 4 schematically illustrates example operations in another process flow 400 for detecting materials in a mixture using oligonucleotides. Process flow 400 illustrated in FIG. 4 may include mixing at least two materials with one another to obtain a mixture, different oligonucleotides having different sequences than one another being disposed within different ones of the materials (operation 410). The materials may be mixed in any suitable reservoir, such as a waste reservoir (e.g., as described with reference to FIGS. 1A-1B) or a mixing reservoir (e.g., as described with reference to FIG. 5. In some examples, the materials may be selected from the group consisting of a liquid, a semi-solid, and a solid. In one nonlimiting example, the materials include reagents for use in sequencing and/or may be provided in a substrate comprising a plurality of reservoirs, each material being within a respective one of the reservoirs, e.g., such as described with reference to FIGS. 1A-1B, although it will be appreciated that any suitable types of materials and arrangement of reservoirs, and manner of mixing the materials, may be used. The oligonucleotides may include single-stranded DNA, although other suitable types of oligonucleotides may be used. In one nonlimiting, purely illustrative example, the mixture is obtained using a first sequencing system.

Process flow 400 illustrated in FIG. 4 may include that the sequences of the oligonucleotides are used to detect the materials in the mixture (operation 420). In a manner such as described with reference to FIGS. 2A-2B, the sequence of each of the oligonucleotides may include an index corresponding to the mixture and/or may include a barcode corresponding to the respective material. In a manner such as described with reference to FIGS. 1A-1B, the materials may be detected by comparing the sequences of the oligonucleotides to stored sequences. Additionally, in some examples, the materials are detected by quantifying respective amounts of the oligonucleotides, and correlating the amounts of the oligonucleotides to amounts of the respective materials. In one nonlimiting, purely illustrative example, the oligonucleotides in the mixture are sequenced using a second sequencing system that is different from the first sequencing system. In such an example, the sequences of the oligonucleotides may include adapters that are compatible with the second sequencing system and are incompatible with the first sequencing system, so as to inhibit sequencing of the oligonucleotides on the first sequencing system.

### WORKING EXAMPLES

Additional examples are disclosed in further detail in the following examples, which are not in any way intended to limit the scope of the claims.

FIGS. 6A-6C schematically illustrate example measurements in a process flow for detecting liquids in a mixture using oligonucleotides. More specifically, a set of oligonucleotides was prepared in which 26 bases of different respective portions of the PhiX genome were used as barcodes and 34 bases of NEXTERA™ PCR tagmentation adapter sequences were provided as adapters on each side in a manner such as illustrated in FIG. 2A. The standard curve illustrated in FIG. 6A was prepared using the internal ladder method described above, in which the percent of identified reads was correlated to oligonucleotide concentration.

Different ones of the oligonucleotides were mixed into each of the liquids within a prefilled cartridge of an iSEQ™ 100 Sequencing System, commercially available from Illumina, Inc. (San Diego, Calif.). The iSEQ™ 100 Sequencing System was used to perform sequencing operations using different liquids therein, and waste from those operations was collected and oligonucleotides therein were amplified and then sequenced. The amount of different liquids in the samples of the waste was determined using the sequences of the oligonucleotides and the amounts of those sequences in the waste, together with the standard curve (FIG. 6A) and the known concentrations of those sequences in the different liquids. More specifically, the known concentrations of the oligonucleotides in the original solutions were used to understand their respective dilutions within the waste pool. For example, if the respective concentrations of oligonucleotides within first and second reagents going into waste are initially the same, and in the sample of the waste pool a first one of the oligonucleotides has 300 reads and the other has 700 reads, then it is determined that the first reagent is 30% of the waste solution. Using the total volume of the waste, this concentration may be converted to volume.

FIG. 6B illustrates the volumes of different liquids that were determined to be in the waste sample from an example process in the iSEQ™ 100 Sequencing System, and Table 1 below lists such volumes. Note that some liquids (such as HCX1, HCX2, and HCX3) each contained multiple oligonucleotides.

TABLE 1

| | | Oligonucleotide | Volume |
|---|---|---|---|
| Liquid | Purpose | name | (nL) |
| HCX1 | Amplifying clusters on flowcell | HCX1-A | 0.81 |
| HCX1 | Amplifying clusters on flowcell | HCX1-B | 0.12 |
| HCX1 | Amplifying clusters on flowcell | HCX1-C | 0.02 |
| HCX1 | Amplifying clusters on flowcell | HCX1-D | 0.01 |
| HCX2 | Amplifying clusters on flowcell | HCX2-A | 0.06 |
| HCX2 | Amplifying clusters on flowcell | HCX2-B | 0.01 |
| HCX2 | Amplifying clusters on flowcell | HCX2-C | 0.01 |
| HCX2 | Amplifying clusters on flowcell | HCX2-D | 0.00 |
| HCX3 | Amplifying clusters on flowcell | HCX3-A | 0.05 |
| HCX3 | Amplifying clusters on flowcell | HCX3-B | 0.01 |
| HCX3 | Amplifying clusters on flowcell | HCX3-C | 0.05 |
| HCX3 | Amplifying clusters on flowcell | HCX3-D | 0.00 |
| HP10 | Sequencing primer mix | HP10 | 1.82 |
| HP11 | Sequencing primer mix | HP11 | 39.74 |
| HP14 | Sequencing primer mix (first well) | HP14-1 | 4.00 |
| HP14 | Sequencing primer mix (second | HP14-2 | 0.00 |
| | well) | | |
| HT1 | Hyb buffer for library dilution | HT1 | 34.80 |
| TMP | Empty template well, for user to | TMP | 0.00 |
| | add template DNA | | |
| WAM | Amplification mix used for the PE | WAM | 8.66 |
| | turn and post exo repair | | |
| WB6 | Wash buffer | WB6 | 0.00 |
| WB7 | Wash buffer | WB7 | 214.12 |
| WCM | SBS cleavage reagent | WCM | 75.91 |
| WDR | Denaturing reagent used in on- | WDR | 45.98 |
| | board library denaturing | | |
| WEX | Exo nuclease reagent used to | WEX | 0.03 |
| | remove unused primers | | |
| WIM-1 | Component of SBS incorporation | WIM-1 | 3222.50 |
| | mix | | |
| WIM-1 | Component of SBS incorporation | WIM-2 | 140.93 |
| | mix | | |
| WLM1 | Linearization reagent for read 1 | WLM1 | 0.01 |
| WPK | Reagent used to clean up proteins | WPK | 40.31 |
| | adhering to flowcell surface | | |
| WPM | Wash buffer used between WAM | WPM | 0.24 |
| | amplification cycles | | |
| WRM | Deblocking reagent that enables PE | WRM | 3.51 |
| | amplification to proceed | | |
| WSM | SBS scan mix used during imaging | WSM | 249.84 |
| WST | Secondary labeling reagent | WST | 109.72 |
| | enabling base differentiation | | |
| | for 1-dye SBS | | |

From FIG. 6B and Table 1, it may be understood that the present oligonucleotides may be used to determine the respective volume of each material in a mixture of several materials (e.g., a mixture of more than 10 liquids, or more than 20 liquids, or more than 30 liquids) with sub-nanoliter precision (e.g., to detect volumes of about 0.1-500 nL, of about 0.1-100 nL of about 0.1-10 nL, of about 0.1-1 nL).

Additionally, the mixing ratios of different liquids were determined. More specifically, FIG. 6C illustrates the percentages of the liquids HCX1, HCX2, and HCX3 that were obtained from a sample of a mixture of the liquids. As shown in FIG. 6C, the “ideal” mix of the three liquids included 31.48% HCX1, 15.74% HCX2, and 31.48% HCX3, but the sample was determined to include 29.03% HCX1, 16.49% HCX2, and 33.18% HCX3. From FIG. 6C, it may be understood that the present oligonucleotides may be used to determine the relative percentage of a mixture that is provided by each material in a mixture, and may be used to assess process improvements for use in bringing the actual mixture of the three materials closer to the “ideal” mix for a desired process, e.g., by increasing or decreasing the amount of one or more of the materials added to the process.

### ADDITIONAL COMMENTS

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

While various illustrative examples are described above, it will be apparent to one skilled in the art that various changes and modifications may be made therein without departing from the invention. The appended claims are intended to cover all such changes and modifications that fall within the true spirit and scope of the invention.

1. A method of detecting materials in a mixture of materials, the method comprising:
providing materials;
providing oligonucleotides having different sequences than one another, each of the oligonucleotides being within, and corresponding to, a respective one of the materials;
obtaining a mixture of at least two of the materials with one another, the mixture comprising the oligonucleotides corresponding to those materials;
sequencing the oligonucleotides in the mixture; and
detecting the materials corresponding to those oligonucleotides using the sequences of those oligonucleotides.

2. The method of claim 1, wherein the materials comprise reagents for use in sequencing.

3. The method of claim 1, wherein the materials respectively are provided in reservoirs of a substrate.

4. The method of claim 1, wherein the sequence of each of the oligonucleotides comprises: (i) an index corresponding to the mixture and (ii) a barcode corresponding to the respective material.

5. The method of claim 1, wherein the sequence of each of the oligonucleotides includes a barcode corresponding to the respective material, and the method further comprising adding, to each of the oligonucleotides, an index corresponding to the mixture.

6. The method of claim 5, wherein the index is added after obtaining the mixture.

7. The method of claim 1, wherein the mixture is obtained using a first sequencing system.

8. The method of claim 7, wherein the mixture comprises waste from the first sequencing system.

9. The method of claim 7, wherein the oligonucleotides in the mixture are sequenced using a second sequencing system that is different from the first sequencing system.

10. The method of claim 9, wherein the sequences of the oligonucleotides comprise adapters that are compatible with the second sequencing system and are incompatible with the first sequencing system.

11. The method of claim 1, wherein sequencing the oligonucleotides in the mixture comprises amplifying the oligonucleotides on a surface to generate respective clusters of amplicons on a surface.

12. The method of claim 1, wherein detecting the materials comprises comparing the sequences of the oligonucleotides to stored sequences.

13. The method of claim 1, wherein detecting the materials comprises quantifying respective amounts of the oligonucleotides, and correlating the amounts of the oligonucleotides to amounts of the respective materials.

14. The method of claim 1, wherein the oligonucleotides comprise single-stranded DNA, double-stranded DNA, RNA, LNA, or a sequence of modified nucleotides.

15. The method of claim 1, wherein each of the materials is independently selected from the group consisting of a liquid, a semi-solid, and a solid.

16. The method of claim 15, wherein the liquid comprises a solvent or reagent.

17. The method of claim 15, wherein the solid comprises a dry powder or a powder in a liquid.

18. The method of claim 15, wherein the semi-solid comprises a gel.

19. A method of detecting a material in a mixture of materials, the method comprising:
mixing at least two materials with one another to obtain a mixture,
different oligonucleotides having different sequences than one another being disposed within different ones of the materials;
wherein the sequences of the oligonucleotides are used to detect the materials in the mixture.

20. The method of claim 19, wherein the materials comprise reagents for use in sequencing.

21. The method of claim 19, wherein the materials respectively are provided in reservoirs of a substrate.

22. The method of claim 19, wherein the sequence of each of the oligonucleotides comprises one or both of (i) an index corresponding to the mixture and (ii) a barcode corresponding to the respective material.

23. The method of claim 19, wherein the mixture is obtained using a first sequencing system.

24. The method of claim 23, wherein the mixture comprises waste from the sequencing system.

25. The method of claim 23, wherein the oligonucleotides in the mixture are sequenced using a second sequencing system that is different from the first sequencing system.

26. The method of claim 25, wherein the sequences of the oligonucleotides comprise adapters that are compatible with the second sequencing system and are incompatible with the first sequencing system.

27. The method of claim 19, to wherein the materials are detected by comparing the sequences of the oligonucleotides to stored sequences.

28. The method of claim 19, wherein the materials are detected by quantifying respective amounts of the oligonucleotides, and correlating the amounts of the oligonucleotides to amounts of the respective materials.

29. The method of claim 19, wherein the oligonucleotides comprise single-stranded DNA, double-stranded DNA, RNA, LNA, or a sequence of modified nucleotides.

30. The method of claim 19, wherein each of the materials is independently selected from the group consisting of a liquid, a semi-solid, and a solid.

31. The method of claim 30, wherein the liquid comprises a solvent or reagent.

32. The method of claim 30, wherein the solid comprises a dry powder or a powder in a liquid.

33. The method of claim 30, wherein the semi-solid comprises a gel.

34. An apparatus, comprising:
a substrate comprising a plurality of reservoirs;
plurality of materials, each of the materials being within a respective one of the reservoirs; and
a plurality of oligonucleotides having different sequences than one another, each of the oligonucleotides being within a respective one of the materials.

35. The apparatus of claim 34, wherein the materials comprise reagents for use in sequencing.

36. The apparatus of claim 34, wherein the sequence of each of the oligonucleotides comprises one or both of (i) an index corresponding to the mixture and (ii) a barcode corresponding to the respective material.

37. The apparatus of claim 34, for use in a sequencing system to obtain a mixture of two or more of the materials.

38. The apparatus of claim 37, wherein the mixture comprises waste from the sequencing system.

39. The apparatus of claim 37, wherein the sequences of the oligonucleotides comprise adapters that are incompatible with the sequencing system.

40. The apparatus of claim 34, wherein the oligonucleotides comprise single-stranded DNA, double-stranded DNA, RNA, LNA, or a sequence of modified nucleotides.

41. The apparatus of claim 34, wherein each of the materials is independently selected from the group consisting of a liquid, a semi-solid, and a solid.

42. The apparatus of claim 41, wherein the liquid comprises a solvent or reagent.

43. The apparatus of claim 41, wherein the solid comprises a dry powder or a powder in a liquid.

44. The apparatus of claim 41, wherein the semi-solid comprises a gel.
